(19) United States
(12) Patent Application Publication
(10) Pub. No.: US 2025/0190498 A1
(43) Pub. Date: Jun. 12, 2025

(54) DOCUMENT CREATION SYSTEM

(71) Applicant: IRYOU JYOUHOU GIJYUTU KENKYUSHO CORPORATION, Fukuoka (JP)

(72) Inventor: Shinkichi HIMENO, Fukuoka (JP)

(73) Assignee: IRYOU JYOUHOU GIJYUTU KENKYUSHO CORPORATION, Fukuoka (JP)

(21) Appl. No.: 18/728,276

(22) PCT Filed: Mar. 8, 2023
(86) PCT No.: PCT/JP2023/008690

§ 371 Date: Jan. 31, 2025

(30) Foreign Application Priority Data
Mar. 13, 2022 (JP) 2022-038735

(51) Int. Cl. G06F 16/93; G06F 40/103; G16H 15/00

(52) U.S. Cl. CPC G06F 16/93; G06F 40/103; G16H 15/00

(57) ABSTRACT

To provide a document creation system capable of flexibly dealing with the system change or the like by limiting modification required at a time of a system change or the like and an influence range thereof within each external application to improve modification work efficiency, and if possible, by allowing in-application modification work to be easily performed at a practical site to significantly reduce modification cost.

A document creation system includes: (i) a core program including a document data management means configured to record and manage document data; and (ii) at least one program (external application) independent of the core program that receives data necessary for processing from (i) and performs processing, in which both of (i) and (ii) cooperate with each other to create a document.

### TECHNICAL FIELD

The present invention relates to a document creation system, particularly a medical document creation system.

### BACKGROUND ART

In a medical site or the like, many documents such as an observation record document, a document instructing a medical act, a document recording a result of an act, and the like are created. Systems that create, record, and refer to documents continue to expand gradually in response to demands for increased medical complexity, for example, ensuring safety to prevent the occurrence of errors. Background art documents related to this application include the following.

### CITATION LIST

### Patent Literature

Patent Literature 1: JP Application No. 2000-309420
Patent Literature 2: JP 3926778 B2

### SUMMARY OF INVENTION

### Technical Problem

Conventional document creation systems have sought to cover everything with a single system. Therefore, a total amount of a program increases, and a development cost and a maintenance cost also increase. In some systems, a mechanism in which modules for doctors, nursing, and the like are divided, developed, and combined as necessary has also been proposed. However, since there is a lack of information cooperation between the modules, it is difficult to obtain information of other industry types, and work efficiency is hindered.

With revision of the law, a type of document to be prepared, items in the document, and a layout of the items are frequently changed. In order to cope with this change, it is necessary to modify software of a main body of the document system, but since there is a possibility that the change affects an entire system, enormous work man-hours are required to prevent creation of a bug. Each time, a large amount of cost is generated, which is a factor that puts pressure on management.

The present invention has been made to solve such a conventional problem, and a document creation system is configured such that (1) a basic portion (hereinafter, referred to as a “core program”) of case management, document creation/editing, document recording, document editing, and the like with relatively few changes and (2) a document output portion with frequent changes are divided into at least one program group (hereinafter, referred to as an “external application” group) independent of the core program. An object of the present invention is to provide a document creation system capable of improving modification work efficiency by limiting modification required at a time of a system change or the like and an influence range thereof within each external application, and if possible, capable of flexibly coping with the system change or the like by dramatically reducing modification cost by enabling in-application modification work to be easily performed at a practical site (so-called digital transformation (DX)).

### Solution to Problem

As a means configured to achieve the object of the present invention, in a document creation system according to claim 1, the document creation system includes: (i) a core program including a document data management means configured to record and manage document data, and (ii) at least one external application independent of the core program for receiving data from (i) and performing processing;
the external application includes a necessary document element data requesting means configured to transmit to the core program a transmission request for document element data that is a part of a document, and includes an external application API for receiving the document element data; and
the core program includes a document list acquisition means configured to acquire a document list from the document data management means according to the necessary document element data requesting means, a specific document data acquisition means configured to acquire necessary specific document data from the document list, and a necessary document element data extraction means extracts necessary document element data from the specific document data according to a request from the necessary document element data requesting means, and the core program creates a document by both of (i) and (ii).

In the document creation system according to claim 2, in the document creation system according to claim 1, the external application API further includes an external application created document data transmission means configured to transmit document data created by the external application to the core program.

In the document creation system according to claim 3, in the document creation system according to claim 1 or 2, the core program includes a document data registering means that receives created document data created by the external application and registers the document data in a document data management means.

In the document creation system according to claim 4, the document creation system according to any one of claims 1 to 3 includes a document format reference means configured to refer to a format of a document to be created in the external application from outside the external application.

In the document creation system according to claim 5, the document creation system according to any one of claims 1 to 4 includes an external application database in which the document list is recorded.

In the document creation system according to claim 6, the document creation system according to any one of claims 1 to 5 includes: a screen display pattern management means configured to, in the document creation system includes, manage a screen display pattern name for each document creation status; a screen display pattern breakdown management means configured to manage a display position and a display size of the screen display pattern on a screen; and a screen display pattern selection means capable of selecting a screen display pattern suitable for each document creation status.

In the document creation system according to claim 7, the document creation system according to claim 6 includes a per-user screen display pattern creation management means in which the screen display pattern management means is capable of creating a screen display pattern for each user.

### Advantageous Effects of Invention

In the document creation system according to claim 1, in the document creation system, (i) the core program including the document data management means configured to record and manage document data, and (ii) at least one program (external application) that receives data necessary for processing from (i), performs processing, and is independent of the core program cooperate with each other to create a document.

In addition, the external application API is provided that transmits the transmission request for document element data required for processing (necessary document element data requesting means) from the external application to the core program and receives the document element data (necessary document element data receiving means).

Furthermore, the core program includes the necessary document element data extraction means, which acquires the document list from the document data management means according to the necessary document element data requesting means (document list acquisition means), acquires the necessary specific document data from the document list (specific document data acquisition means), and extracts the necessary document element data from the specific document data according to the request from the necessary document element data requesting means.

Since the document creation system according to claim 2 includes the external application created document data transmission means, the external application API further transmits the document data created by the external application to the core program.

Since the document creation system according to claim 3 includes the document data registering means, the created document data created by the external application is received, and the document data is registered in the document data management means.

Since the document creation system according to claim 4 includes the document format reference means, the format of the document to be created is referred to from outside the application.

Since the document creation system according to claim 5 includes an external application database management means, the application itself manages a database necessary for processing.

The document creation system according to claim 6 includes: the screen display pattern management means configured to manage the screen display pattern name for each document creation status, the screen display pattern breakdown management means configured to manage the display position and the display size of an output of the external application or the core program displayed for each of the screen display patterns, and the screen display pattern selection means capable of selecting a screen display pattern suitable for each document creation status.

Since the document creation system according to claim 7 includes the per-user screen display pattern creation management means, a screen display pattern can be created for each user.

### BRIEF DESCRIPTION OF DRAWINGS

FIG. 1 is a hardware configuration diagram of the present invention.

FIG. 2 is a software configuration diagram illustrating a relationship between a core program and an external application group.

FIGS. 3(a), 3(b), and 3(c) illustrate a mode of data management in the core program.

FIG. 3(a) is an example of a case ID management means.

FIG. 3(b) is an example of a document category management means.

FIG. 3(c) is an example of a document category section management means.

FIG. 3(d) is an example of a document data management means.

FIG. 4 is an explanatory diagram of a document data input/output means.

FIG. 5 is an explanatory diagram of an external application data input/output means.

FIG. 5(a) illustrates a processing flow when outputting a document element required by an external application.

FIG. 5(b) illustrates a processing flow when document data created by the external application is registered in the document data management means of the core program.

FIG. 6(a) illustrates an example of creating a new document by the external application.

FIG. 6(b) illustrates another example of creating a new document by the external application.

FIG. 7 illustrates a preferred screen display mode of the present invention.

### DESCRIPTION OF EMBODIMENT

A document creation system according to the present invention includes a server device, a database, and a terminal.

The server device is a known computer device, and includes an arithmetic device, a main storage device, an auxiliary storage device, an input device, an output device, and a communication device.

The arithmetic device, the main storage device, the auxiliary storage device, the input device, the output device, and the communication device are connected to each other via a bus interface.

The arithmetic device includes a known processor capable of executing a command set.

The main storage device includes a volatile memory such as a RAM capable of temporarily storing the command set.

The auxiliary storage device includes a non-volatile data storage capable of recording an OS and a program.

The data storage may be, for example, an HDD or an SSD.

The input device is, for example, a keyboard.

The output device is, for example, a display such as an LCD.

The communication device includes a network interface connectable to a network.

The server device has means such as a document data management means, a necessary document element data requesting means, a necessary document element data receiving means, a document list acquisition means, a specific document data acquisition means, a necessary document element data extraction means, an external application created document data transmission means, a document data registering means, a document format reference means, an external application database management means, a screen display pattern management means, a screen display pattern breakdown management means, a screen display pattern selection means, a per-user screen display pattern creation management means, and an external application form providing means.

The processor of the server device exerts a functional effect of the means.

The database according to the present invention may be configured by the auxiliary storage device of the server device, or may be configured by another auxiliary storage device independent of the server device.

The database stores information handled by the document creation system.

The terminal according to the present invention has a hardware configuration of the known computer similarly to the server device.

The server device, the database, and the terminal according to the present invention can communicate via the network.

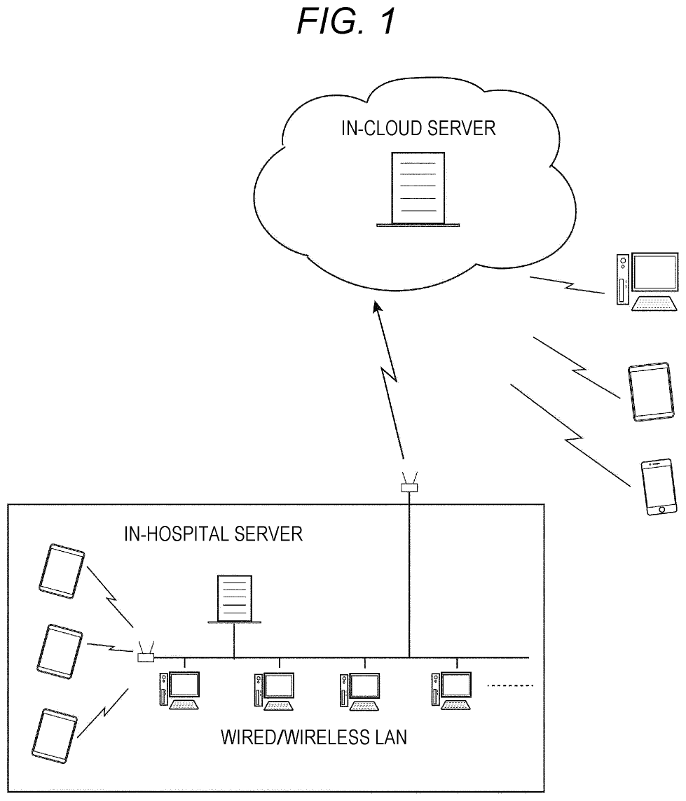

FIG. 1 illustrates a hardware configuration diagram of the present invention.

An in-hospital server is in a facility, and the document creation system of the present invention is operating. Terminals in the hospital are connected by a wired or wireless LAN. The terminals include a device group such as a PC, a tablet, and a smartphone. It is also possible to install a main or a backup server on the cloud as necessary.

Devices outside the hospital can also access the in-hospital server or the in-cloud server via a web line or a public line.

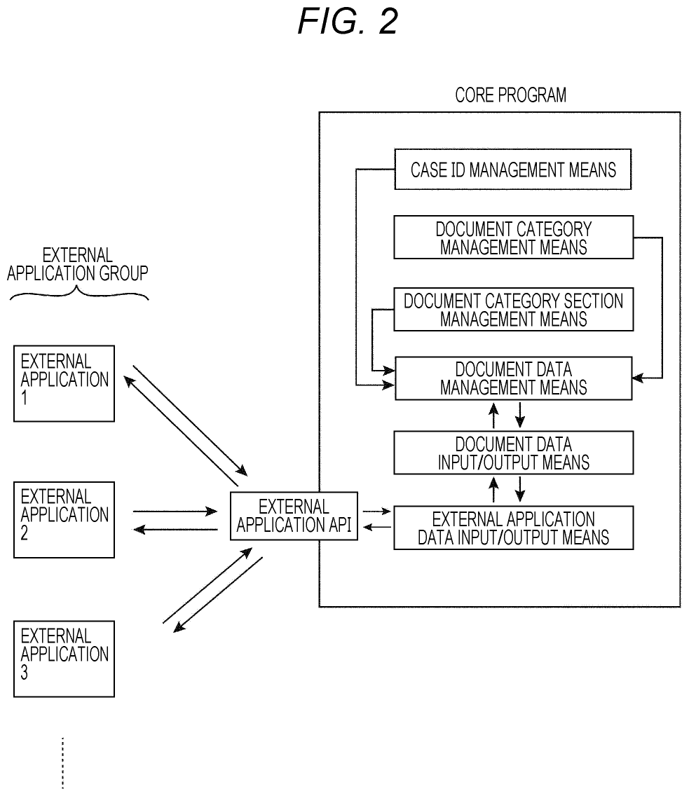

FIG. 2 is a software configuration diagram illustrating a relationship between a core program and an external application group.

Included are the core program such as case ID management, document creation/editing, document recording, and document editing with a relatively small number of changes, and at least one external application group that is independent of the core program and is responsible for a frequently changed document output portion, and both are configured as software linked by an external application API.

API is an abbreviation of “application programming interface”, and is a series of definitions and communication protocols for constructing and integrating data cooperation between application software.

The core program includes a case ID management means configured to manage an ID (patient ID in a medical institution) for distinguishing cases, a type (category) of a document created for each case, a document category section management means configured to manage a section described in the document category, a document data management means configured to record and searches document data for each case ID, each document category, and each section in the document category, and a document data input/output means having functions of creating new document data, editing existing document data, and displaying and printing document data (the above is a configuration of a standard core program of the document creation system). The document category section management means is not necessarily required when a structure of the document category is flat and sectioning is not required.

The present invention includes an external application data input/output means configured to further extract necessary document element data from the document data and transmit the document element data to an application via the external application API, and conversely, receive the document data transmitted from the application, display content, and record the document data in the document data management means via the document data input/output means as necessary.

The external application performs various types of processing using the document element data received via the external application data input/output means and the external application API, and displays and prints a processing result. A new document of the processing result created as necessary is transmitted via the external application API to the external application data input/output means in the core program. The core program registers the received document data in the document data management means as necessary.

The external application group may be in the same device as the core program, or a part or all of the external application group may be in another independent device. Each application may be resident in a device (resident type external application) or may be distributed to the device as necessary and deleted after operation (temporary external application). Communication between the external application and the core program in the external application API may be selectively used as necessary, such as via a memory in the device, via a communication line, or via a temporary file.

When a plurality of staff members shares the same device, there is a risk that it becomes unclear in which case the external application cooperates with the core program. In order to prevent this risk, it is necessary to transmit a case ID and designation of an application to be activated from the core program via the external application API, and to activate the designated application only for the designated case ID (in the case of the temporary external application).

When the designated application is a resident type application, it is necessary to individually manage the case ID for each staff member sharing the core program so as not to confuse the case data for which the staff member is responsible.

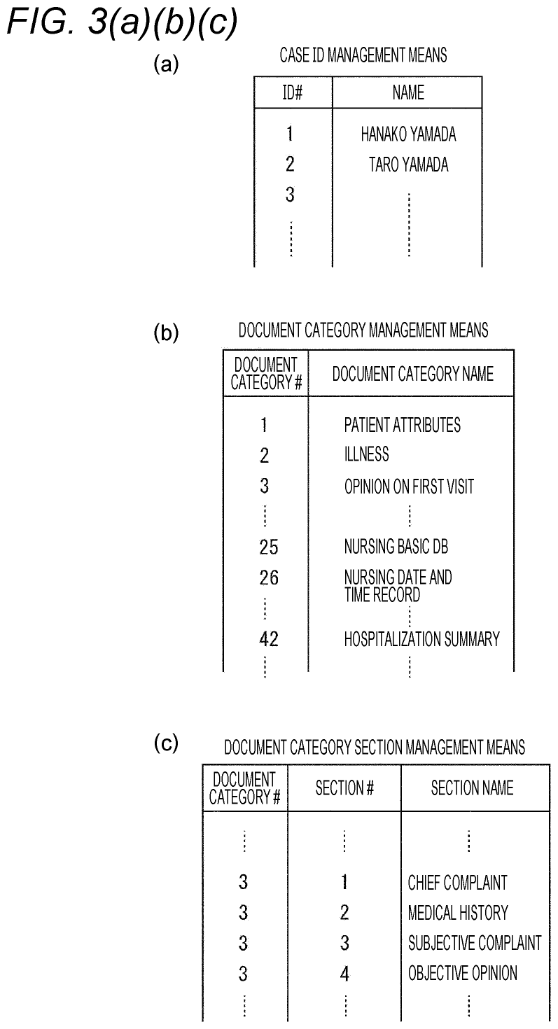

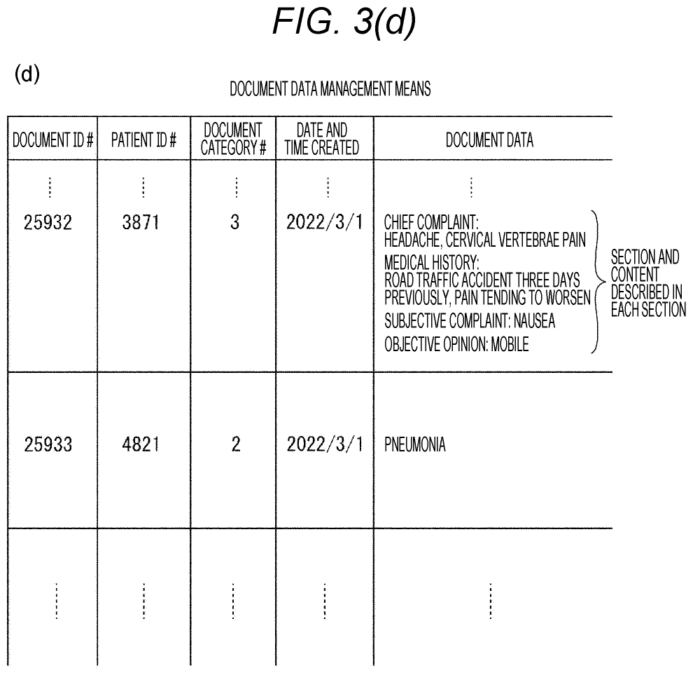

FIGS. 3(a) to 3(d) illustrate a mode of a data management means in the core program.

FIG. 3(a) is an example of the case ID management means.

A list of case ID numbers (patient ID numbers in the case of a medical institution) is managed.

The document data is managed in association with the case ID.

FIG. 3(b) is an example of a document category management means.

The document data related to a case is organized and recorded for each document category number defined in the document category management means.

FIG. 3(c) is an example of the document category section management means.

A section number provided inside is defined for each document category, and document data corresponding to each section number is recorded.

FIG. 3(d) is an example of the document data management means.

A document ID number is assigned to each document, and document data is organized and recorded for each section inside the document together with a case ID number (patient ID number in this example) to which the document belongs and a document category number.

In this example, the data in all the sections is collectively recorded, but the document data may be divided for each section and recorded as separate rows (records). Although a section name is clearly indicated at the beginning of the row in this example, it is not always necessary to clearly indicate the section name as long as the section name can be specified by an implicit method.

In FIGS. 3(a) to 3(d), document data management is performed using a tabular form of a relational database, but a network type or a hierarchical type database such as CODASYL may be used in some cases.

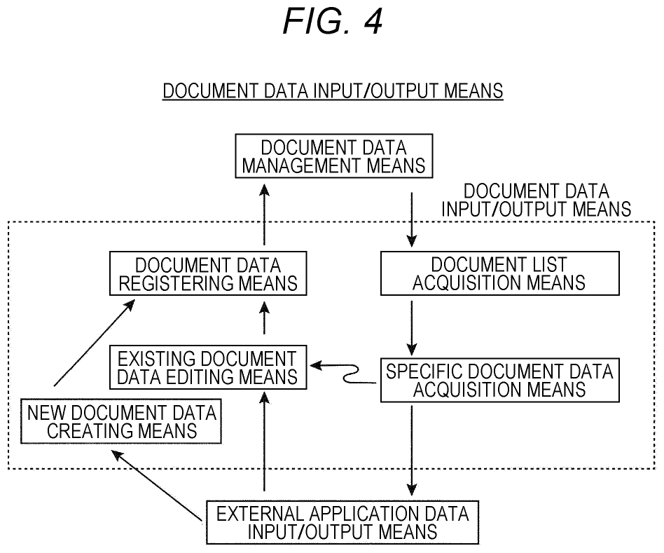

FIG. 4 is an explanatory diagram of the document data input/output means.

From the document data management means as illustrated in FIG. 3, a list of documents matching a search condition is sought, such as by a document list of a certain document category being obtained (document list acquisition means), and a specific document is selected from the list to acquire document data of the document (specific document data acquisition means). The acquired document data may be displayed or printed in the core program, or may be output to the external application via the external application data input/output means.

In addition, an existing document editing means performs editing such as addition and change of document data content, and a document registering means reflects the edited content in the document.

When a new document is created in a designated document category (new document creating means), the created new document data is registered in the document data management means via the document registering means.

Furthermore, the document data created by the external application is registered in the document data management means via the new document creating means and the document registering means. In a case where the document data created by the external application or the existing document data is edited, the document data is registered in the document data management means via the existing document editing means and the document registering means.

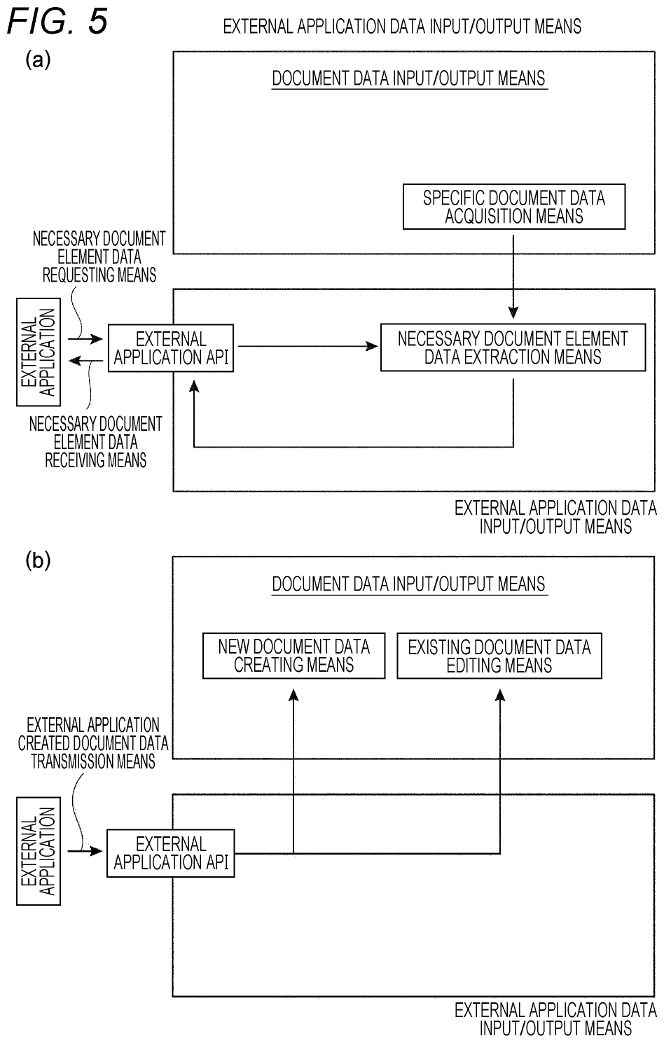

FIG. 5 is an explanatory diagram of the external application data input/output means.

FIG. 5(a) illustrates a processing flow when outputting a document element required by the external application.

Via the external application API, the external application requests the external application input/output means to extract the document element necessary for processing in the external application from the document data acquired by the specific document data acquisition means (necessary document element data extraction requesting means).

The extracted document element data is transmitted to the external application that has requested the document element data via the external application API (necessary document element data receiving means).

The external application receives document element data from a plurality of documents as necessary for processing, performs processing, and displays and prints a processing result as independent processing of the application. Alternatively, a new document is created and transmitted to the external application data input/output means (created document data transmission means).

In some cases, the existing document data created by the application may be received from the core program, edited, and then transmitted to the core program to correct the document data.

Here, the document element means a cluster of data required for the processing in the document data. A size of the document element (data granularity) may be arbitrarily specified, such as an entire document, an entire specific section in the document, or a specific word/phrase tagged in the section.

The tagging may be explicitly performed, for example, “name: Taro Yamada”, or the tag “body temperature” may be implicitly extracted from “body temperature was 38.5° C.” and the like, and then “38.5° C.” may be used as the document element.

In the case of the temporary external application, since the document element data to be transmitted from the core program to the external application is obvious, the necessary document element data extraction requesting means may be omitted.

FIG. 5(b) illustrates a processing flow when the document data created by the external application is registered in the document data management means of the core program.

The document data is transmitted to the core program by the external application created document data transmission means via the external application API, passed to the new document data creating means or the existing document data editing means of the document data input/output means, and registered in the document data management means.

Note that, in FIGS. 5(a) and 5(b), in order to avoid complexity of the drawing, only portions necessary for description of processing are displayed inside the document data input/output means and the external application data input/output means.

FIGS. 5(a) and 5(b) illustrate an example of cooperation between the core program and the external application, and the present invention is not limited to this configuration.

If it is possible to extract necessary document elements from the core program and transmit the extracted document elements to the external application via the external application API, and to transmit and register document data created/edited as the processing result of the external application to the core program via the external application API, the means group can be changed to an arbitrary configuration.

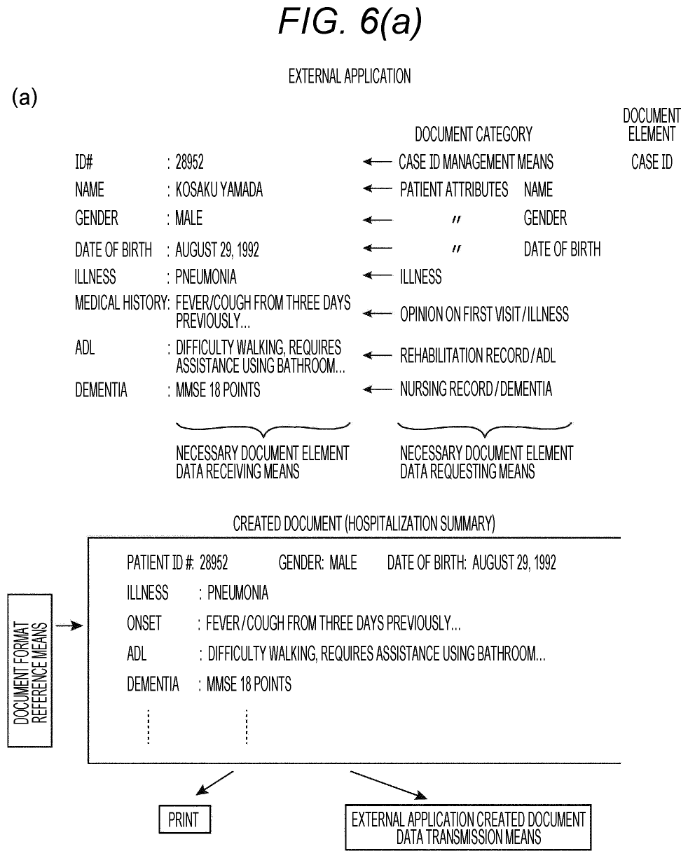

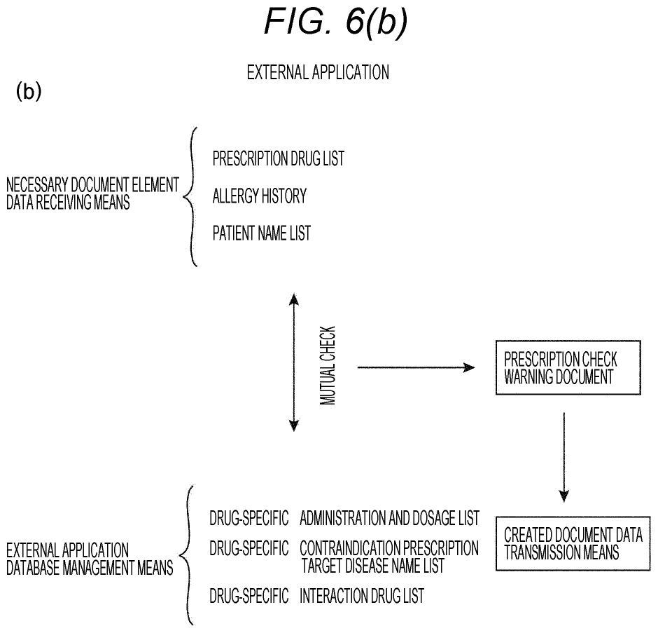

FIG. 6(a) illustrates an example in which a new document is created by the external application.

The external application transmits a request for document element data necessary for processing to the core program via the external application API (necessary document element data requesting means).

Requested document element data necessary for processing is received from the core program (necessary document element data receiving means), document elements are arranged according to a format determined in the external application, and a new document (hospitalization summary in this example) is created.

The created new document may be displayed and printed to complete the processing in the external application, or may be transmitted to the core program via the external application API (external application created document data transmission means) and registered in the document data management means.

The format determined in the external application may be directly described in the external application, or a format defined in an appropriate spreadsheet, a standard document, or the like may be called from outside the external application (document format reference means).

When it is necessary to change the format due to revision of the law or the like, it can be said that the latter is a more preferable mode since it is only necessary to change the description of the format defined in the spreadsheet, the standard document, or the like without changing the external application itself.

FIG. 6(b) illustrates another example of creating a new document by the external application.

The external application receives a prescription drug list, an allergy history, and a disease name list from the core program. The external application manages databases such as a drug-specific administration and dosage list, a drug-specific contraindication prescription target disease name list, and a drug-specific interaction drug list (external application database management means).

These databases may be constructed in the external application, or necessary data may be appropriately read as an independent database group.

The latter is a more preferable mode because an update of the database is convenient.

The received prescription drug list, allergy history, and disease name list are collated with information obtained from the database, and it is confirmed whether an administrated dosage of each drug is within an appropriate range, whether the drug is affected by a contraindicated disease, whether there is an interaction such as enhancement or attenuation of action between prescribed drugs, and the like.

If there is content for which a warning should be provided, the content is listed, and a prescription check warning document is created and displayed.

A prescription is often changed according to the warning, but even if there is a warning, the prescription may be intentionally issued at the discretion of a doctor.

At this time, a confirmation input of the doctor who intentionally issues the prescription is requested for a warning message, the prescription check warning document including the confirmation is created, and the external application created document data transmission means transmits and registers the prescription check warning document in the core program. In this way, an attending physician's intention becomes clear even for staff in the field such as a pharmacist, and thus, work of inquiring about an irregular prescription becomes unnecessary.

In the development of external applications, there are many common parts such as management of user IDs, security, and a method of communication with the core program via the external application API.

If these common parts are drawn up and defined as a framework so that the external application can be developed if only an operation specific to the application is described (software development kit (SDK)), even a staff member on site who has specialized knowledge in the field but has little software development experience can easily develop the external application.

This makes it possible to develop and maintain an entire document creation system at low cost.

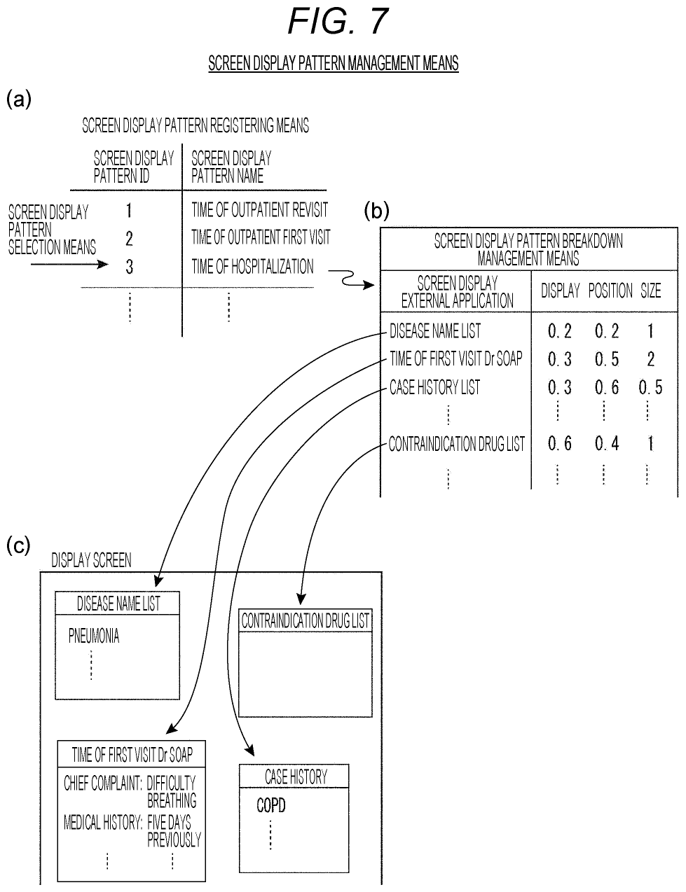

FIG. 7 shows the screen display pattern management means, which is a preferred screen display mode of the present invention.

Information necessary for creating and referring to a document, such as an outpatient first visit, an outpatient revisit, a time of hospitalization, a course after hospitalization, and a time of creating a daily nursing record, is different.

When the necessary information is scattered in a plurality of document categories, work can be efficiently performed as long as documents of the related document categories can be simultaneously displayed on a screen.

(a) The screen display pattern registering means registers a screen display pattern according to a work situation (scene). An appropriate screen display pattern is selected as necessary (screen display pattern selection means).

(b) The screen display pattern breakdown management means defines the external application that displays each document and a display position and size on the screen. If necessary, display of the core program is also used.

(c) is a display screen on the device. For display of each application, a program may be written in a unique language such as BASIC or COBOL, but it is more desirable to display each application on a web browser using HTML, which is currently used as standard.

This is because display can be performed regardless of a device such as a normal personal computer, a tablet, or a smartphone, and various support programs related to the web browser can be used together.

The registration of (a) and (b) may be shared by an entire organization or may be additionally defined by individual users.

Although the embodiment has been described above, a specific configuration of the present invention is not limited to the embodiment, and design changes and the like that do not depart from the gist of the invention are included in the present invention.

For example, which function is allocated to the external application group among functions of a conventional core program is arbitrary. The core program and the external application group are normally operated on the same device, but some external applications may be operated on different devices.

In addition, although medical care has been described as an example in the present specification, applications in other fields are also included in the present invention.

1. A document creation system comprising:
(i) a core program including a document data management means configured to record and manage document data; and
(ii) at least one external application that receives data from (i) and performs processing, the external application being independent of the core program, wherein
the external application includes a necessary document element data requesting means configured to transmit a transmission request for document element data that is a part of a document to the core program, and includes an external application API for receiving the document element data, and
the core program includes a document list acquisition means configured to acquire a document list from the document data management means according to the necessary document element data requesting means, a specific document data acquisition means configured to acquire necessary specific document data from the document list, and a necessary document element data extraction means configured to extract necessary document element data from the specific document data according to the request from the necessary document element data requesting means, and the core program creates a document by both of (i) and (ii).

2. The document creation system according to claim 1, wherein the external application API further includes an external application created document data transmission means configured to transmit document data created by the external application to the core program.

3. The document creation system according to claim 1 or 2, wherein the core program includes a document data registering means configured to receive created document data created by the external application and register the document data in the document data management means.

4. The document creation system according to any one of claims 1 to 3, comprising a document format reference means configured to refer to a format of a document to be created in the external application from outside the external application.

5. The document creation system according to any one of claims 1 to 4, comprising an external application database in which the document list is recorded.

6. The document creation system according to any one of claims 1 to 5, comprising: a screen display pattern management means configured to manage a screen display pattern name for each document creation status; a screen display pattern breakdown management means configured to manage a display position and a display size of the screen display pattern on a screen, and a screen display pattern selection means capable of selecting a screen display pattern suitable for each document creation status.

7. The document creation system according to claim 6, wherein the screen display pattern management means includes a per-user screen display pattern creation management means capable of creating a screen display pattern for each user.
